(19) United States
(12) Patent Application Publication
(10) Pub. No.: US 2018/0296153 A1
(43) Pub. Date: Oct. 18, 2018

(54) URINATION VOLUME MANAGEMENT SYSTEM

(71) Applicant: SHIMADZU CORPORATION, Kyoto (JP)

(72) Inventor: Nobuya HASHIZUME, Kyoto (JP)

(21) Appl. No.: 15/949,871

(22) Filed: Apr. 10, 2018

(30) Foreign Application Priority Data
Apr. 12, 2017 (JP) 2017-078743

(51) Int. Cl. A61B 5/20; G01F 22/00; G01G 19/50; A61B 5/00

(52) U.S. Cl. CPC A61B 5/204; G01F 22/00; A61B 5/0022; A61B 5/7203; A61B 5/7278; G01G 19/50

(57) ABSTRACT

A voided volume calculation unit automatically calculates a voided volume of a subject based on information for a weight value before voiding and information for the weight value after voiding that have been transmitted from a weight measurement unit. The memory unit takes the calculated voided volume of the subject as voided volume information, and makes association with additional information regarding voiding accepted by an additional information input accepting unit. Then, the memory unit records the pieces of information associated with each other as the voiding information. Therefore, it is possible to perform detailed management of the information regarding voiding.

### BACKGROUND OF THE INVENTION

### Field of the Invention

The present invention relates to a voided volume management system used to manage information on voided volumes of a subject.

### Description of the Related Art

Management of information related to voided volumes may be performed for a subject with various voiding symptoms such as dysuria. Specifically, each time upon voiding, a subject passes urine in a container, and measures a voided volume using a scale on the container. Then, the subject records a volume and time and date for each voiding on recording paper, and submits the record to an examiner such as a medical practitioner. Checking the recording paper submitted by the subject allows the examiner to observe fluctuations in symptoms of the subject, and to deal with the symptoms appropriately.

However, it is annoyingly troublesome for the subject to pass urine in a container each time upon voiding. For this reason, a voided volume management system has been known conventionally, which is used to measure information relating to the subject, and calculates and manages the voided volumes based on the measurement result.

As this type of voided volume management systems, a voided volume management system has been proposed, which includes: a weight measurement unit for measuring changes in the weight of a subject during voiding; a timer unit for keeping time for voiding; and a voiding calculation unit for calculating a voided volume (e.g., JP-A-2002-345793).

According to this voided volume management system, the voiding calculation unit calculates a voided volume of the subject based on data of an amount of weight change during voiding measured by the weight measurement unit and of time for voiding counted by the timer unit. JP-A-2002-345793 also discloses a configuration with which a total voided volume is obtained based on the weight before and after the voiding.

Similarly, a voided volume management system has been proposed as this type of voided volume management systems, which includes: a water level measuring unit for measuring a water level within a toilet bowl; and a urine output calculating unit for calculating a voided volume (e.g., JP-A-2006-219977).

According to this voided volume management system, the urine output calculating unit calculates a voided volume of the subject based on a measurement value of the water level measured by the water level measuring unit.

### SUMMARY OF THE INVENTION

While the conventional voided volume management system described above enables management of information regarding voided volumes without troubles for a subject, it also has a problem that detailed management of other information regarding voiding is not possible. Specifically, with the method in which a subject records voided volumes and voiding time and date on recording paper and submits the record to the examiner, the subject is able to additionally write other information regarding voiding on the recording paper, and the examiner is able to deal in consideration of the information appropriately. However, the conventional voided volume management system described above has a problem that it is not possible to carry out detailed management of information regarding voiding other than voided volumes that is useful for testing.

The present invention is made in view of the above circumstances, and aims to provide a voided volume management system that enables detailed management of information regarding voiding.

(1) A voided volume management system according to the present invention is a voided volume management system for management of voided volumes of a subject, and includes: a weight measurement unit, a voided volume calculation unit, an additional information input accepting unit, and a memory unit. The weight measurement unit measures a weight of a subject before and after voiding. The voided volume calculation unit calculates a voided volume based on the measurement result by the weight measurement unit. The additional information input accepting unit accepts an input of additional information regarding voiding. The memory unit records the additional information the input of which is accepted by the additional information input accepting unit, in association with the voided volume calculated by the voided volume calculation unit.

According to the configuration described above, the voided volume calculation unit automatically calculates the voided volume of the subject based on the measurement result of the weight of the subject before and after voiding. The memory unit records the calculated voided volume of the subject and the input additional information in association with the voided volume.

Therefore, it is possible to carry out detailed management of the information regarding voiding.

(2) Further, the additional information may include at least one piece of: information on whether or not the voiding is carried out during outing, information on whether or not the voiding includes a feces, information on evaluation of the voided volume by the subject, information on whether or not there is a desire to void before voiding, and information on whether or not there is urinary incontinence before voiding.

According to the configuration described above, it is possible to carry out management of information useful for testing as the additional information regarding voiding. The information on whether or not the voiding is carried out during outing and the information on whether or not the voiding includes a feces are useful as information indicating whether or not it is difficult for the voided volume calculation unit to calculate the voided volume. The information on evaluation of the voided volume of the subject is useful as information for associating the sense of the subject with an actual voided volume. The information on whether or not there is a desire to void before voiding, and the information on whether or not there is urinary incontinence before voiding are useful as information on conditions before voiding that can affect the test result.

(3) Moreover, the voided volume management system may further include an information reading unit and an estimated value record processing unit. The information reading unit reads previous voided volumes recorded in the memory unit, when an input of information indicating that the voiding is carried out during outing or information indicating that there is a feces during voiding is accepted as the additional information by the additional information input accepting unit. The estimated value record processing unit is configured to record, as an estimated value, one of a voided volume during outing and a voided volume when there is a feces, in the memory unit, based on the previous voided volumes read by the information reading unit.

According to the configuration described above, when the calculation of the voided volume by the voided volume calculation unit is difficult as in the case in which the voiding is carried out during outing, or in which the voiding includes a feces, the voided volume during outing, or the voided volume when there is a feces are recorded as the estimated value in the memory unit, based on the past voided volume recorded in the memory unit.

Therefore, even when the calculation of the voided volume by the voided volume calculation unit is difficult, the information regarding the voided volume may be recorded in the memory unit, it is possible to perform detailed management of the information regarding voiding.

(4) Furthermore, when an input of the evaluation of the voided volume by the subject is accepted as the additional information by the additional information input accepting unit, along with the information indicating that the voiding is carried out during outing or the information indicating that there is a feces during voiding, the information reading unit may read previous voided volumes recorded in the memory unit in association with the same evaluation.

According to the configuration described above, when the calculation of the voided volume by the voided volume calculation unit is difficult, the information reading unit reads, out of the past voided volumes recorded in the memory unit, information whose voided volume is close to an actual voided volume based on the sense of the subject, and records the read value as the estimated value.

Therefore, even when the calculation of the voided volume by the voided volume calculation unit is difficult, it is possible to perform accurate management of the information regarding voiding.

(5) Further, the estimated value record processing unit may calculate an average value of the previous voided volumes read by the information reading unit, and records the average value as the estimated value in the memory unit.

According to the configuration described above, the estimated value record processing unit may calculate a value close to an actual voided volume using the past voided volumes accumulated in the memory unit, and record the calculated value as the estimated value in the memory unit.

Therefore, it is possible to perform accurate management of the information regarding voiding.

(6) Furthermore, the voided volume management system may include an identification information input accepting unit. The identification information input accepting unit accepts inputs of identification information from a plurality of subjects. The memory unit may record a voided volume and additional information in association with the identification information of each of the subjects input of which is accepted by the identification information input accepting unit.

According to the configuration described above, even when there is more than one subject, the voided volumes and the additional information may be managed for each subject.

Therefore, it is possible to perform detailed management of the information regarding voiding for each subject.

(7) Further, the voided volume management system may include a terminal device. The terminal device is configured separately from the weight measurement unit, and capable of communicating with the weight measurement unit. The additional information input accepting unit may be provided in the terminal device.

According to the configuration described above, the terminal device may be carried independently.

Therefore, even when the subject goes out, by carrying the terminal device, it is possible to use the voided volume management system outside.

According to the present invention, in addition to a voided volume of a subject calculated by the voided volume calculation unit, additional information that has been input is recorded in association with the voided volume in the memory unit, and thus detailed management of information regarding voiding is allowed.

### BRIEF DESCRIPTION OF THE DRAWINGS

FIG. 1 is a schematic diagram illustrating a voided volume management system according to one embodiment of the present invention;

FIG. 2 is a block diagram illustrating a specific configuration of a subject-side terminal in FIG. 1;

FIG. 3 is a flowchart showing one example of processing by a control unit of the subject-side terminal, in normal times;

FIG. 4 is a flowchart showing one example of processing by a control unit of the subject-side terminal when the subject is going out; and

FIG. 5 is a flowchart showing one example of processing by a control unit of the subject-side terminal when the subject has a bowel movement.

### DETAILED DESCRIPTION OF THE PREFERRED EMBODIMENTS

### 1. Overall Configuration of Voided Volume Management System

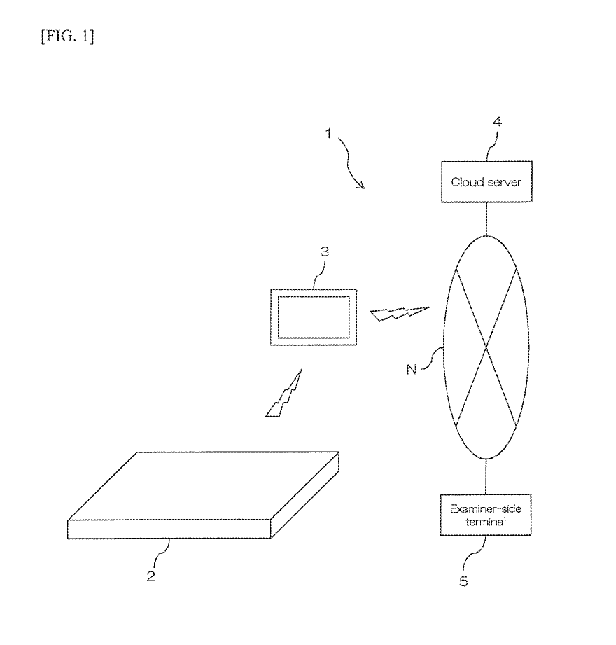

FIG. 1 is a schematic diagram illustrating a voided volume management system 1 according to one embodiment of the present invention.

The voided volume management system 1 includes a weight measurement unit 2, a subject-side terminal 3 as one example of a terminal device, a cloud server 4 enabled to communicate with the subject-side terminal 3 via a network N, and an examiner-side terminal 5 enabled to communicate with the cloud server 4 via the network N.

The weight measurement unit 2 is configured, for example, by a known weight scale, in a box shape. The weight measurement unit 2 is configured to measure a weight of a subject subjected to a test using the voided volume management system 1 when the subject comes on top of the weight measurement unit 2. The weight measurement unit 2 includes a transmission unit (not shown), and is configured to have the transmission unit wirelessly transmit information for a weight value that has been measured to the subject-side terminal 3. The weight measurement unit 2 is normally provided near a lavatory in the subject's home.

The subject-side terminal 3 is configured, for example, by a tablet terminal, separately from the weight measurement unit 2. As will be described later, the subject-side terminal 3 is configured to receive the information for the weight value transmitted from the weight measurement unit 2. Specifically, the subject-side terminal 3 is able to communicate with the weight measurement unit 2. Further, as will be described later, the subject-side terminal 3 is configured to calculate a voided volume based on the weight value, and to accept an input of additional information relating to the voided volume. The subject-side terminal 3 is configured to transmit the calculated voided volume, the additional information, and the like to the cloud server 4 via the Internet N.

The cloud server 4 is configured to record the information transmitted from the subject-side terminal 3.

The examiner-side terminal 5 is configured, for example, by a device of various types such as a personal computer or a tablet terminal. The examiner-side terminal 5 is configured to read the information recorded in the cloud server 4 via the Internet N.

In management of a voided volume using the voided volume management system 1, the subject first stands on the weight measurement unit 2 before voiding. The weight measurement unit 2 measures the weight of the subject before voiding, and transmits the measured weight value to the subject-side terminal 3.

Then, after voiding in the lavatory, the subject again stands on the weight measurement unit 2, and inputs additional information relating to voiding to the subject-side terminal 3. The weight measurement unit 2 measures the weight of the subject after voiding, and transmits the measured weight value to the subject-side terminal 3.

Then, the subject-side terminal 3 calculates a voided volume based on the weight values before and after voiding. Further, the subject-side terminal 3 transmits the calculated voided volume and additional information to the cloud server 4 via the Internet N. The cloud server 4 records the voided volume and the additional information that have been received.

An examiner such as a medical practitioner reads the voided volume and the additional information recorded in the cloud server 4 via the Internet N, using the examiner-side terminal 5. Then, the examiner checks the voided volume and the additional information of the subject on the examiner-side terminal 5, and observes fluctuations in symptoms of the subject based on the information that has been checked.

As described above, according to the voided volume management system 1, as the examiner is able to check, using the examiner-side terminal 5, the additional information regarding voiding that is useful for testing in addition to the voided volume of the subject, the examiner is able to carry out a test appropriately. Further, as will be described later, in the voided volume management system 1, the subject-side terminal 3 calculates the voided volume according to the input additional information.

### 2. Specific Configuration of Subject-Side Terminal

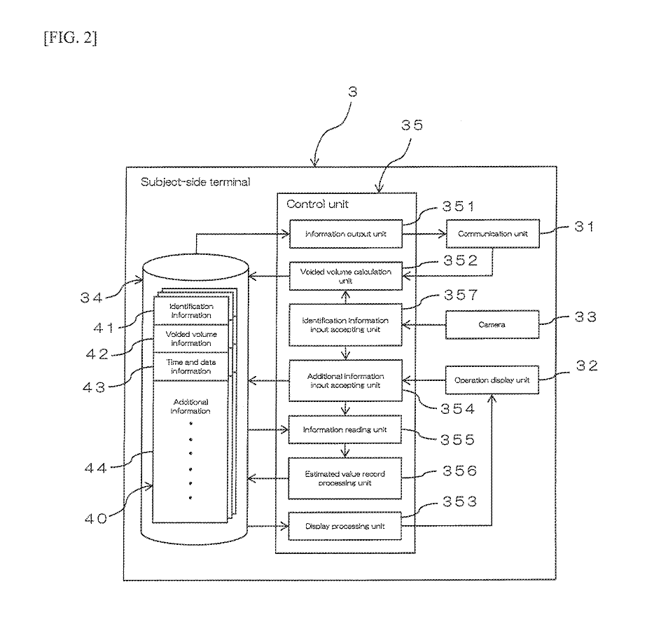

FIG. 2 is a block diagram illustrating a specific configuration of the subject-side terminal 3 in FIG. 1.

The subject-side terminal 3 includes a communication unit 31, an operation display unit 32, a camera 33, a memory unit 34, and a control unit 35.

The communication unit 31 is configured to wirelessly transmit and receive information. Specifically, the communication unit 31 is configured as a reception unit for receiving information from the weight measurement unit 2, and as a transmission unit for transmitting information to the cloud server 4 via the Internet N.

The operation display unit 32 is configured, for example, by a touch panel.

The camera 33 is configured, for example, by a CCD camera or a CMOS camera, and is configured to read a bar code. Specifically, the camera 33 is configured such that information of a bar code is input as identification information. However, the bar code may be read using a bar-code reading terminal or the like.

The memory unit 34 is configured, for example, by a ROM (Read Only Memory), RAM (Random Access Memory), and a hard disk. The memory unit 34 records a plurality of pieces of voiding information 40.

Each piece of the voiding information 40 includes identification information 41, voided volume information 42, time and date information 43, and additional information 44.

The identification information 41 is information for identifying the subject, and different for each subject.

The voided volume information 42 is information for a voided volume of the subject.

The time and date information 43 is information for time and date of voiding by the subject.

The additional information 44 is information regarding voiding of the subject, and useful information for testing. As will be described later, the additional information 44 includes information on whether or not the voiding is carried out during outing, information on whether or not the voiding includes a feces, information on evaluation of the voided volume by the subject, information on whether or not there is a desire to void before voiding, and information on whether or not there is urinary incontinence before voiding.

As described above, in each piece of the voiding information 40, the identification information 41, the voided volume information 42, the time and date information 43, and the additional information 44 are associated with each other.

The control unit 35 includes a CPU (Central Processing Unit), for example. The control unit 35 is able to perform input or output electrical signals among the communication unit 31, the operation display unit 32, and the camera 33. The control unit 35 performs input and output of data, as needed, with the memory unit 34. By having the CPU to perform programs, the control unit 35 may serve, for example, as an information output unit 351, a voided volume calculation unit 352, a display processing unit 353, an additional information input accepting unit 354, an information reading unit 355, an estimated value record processing unit 356, and an identification information input accepting unit 357.

The information output unit 351 reads the voiding information 40 recorded in the memory unit 34, and transmits the voiding information 40 that has been read to the cloud server 4 from the communication unit 31.

The voided volume calculation unit 352 receives information from the weight measurement unit 2 via the communication unit 31, and calculates the voided volume of the subject based on the received information. Specifically, the voided volume calculation unit 352 calculates the voided volume of the subject based on the weight value of the subject that has been received by the communication unit 31, and stores the information on the calculated voided volume in the memory unit 34 as the voided volume information 42.

The display processing unit 353 controls display of the operation display unit 32.

The additional information input accepting unit 354 accepts the additional information input to the operation display unit 32. Then, the additional information input accepting unit 354 stores the accepted additional information 44 in the memory unit 34.

The information reading unit 355 reads the voiding information 40 recorded in the memory unit 34, based on the additional information that has been accepted by the additional information input accepting unit 354.

The estimated value record processing unit 356 calculates the voided volume as an estimated value, based on the voiding information 40 that has been read by the information reading unit 355. Then, the estimated value record processing unit 356 stores the calculated estimated value in the memory unit 34.

The identification information input accepting unit 357 accepts the identification information that has been input via the camera 33. The voided volume calculation unit 352 and the additional information input accepting unit 354 store the voided volume information 42 and the additional information 44 in the memory unit 34, in association with the identification information 41 accepted by the identification information input accepting unit 357.

Further, the subject-side terminal 3 also includes a timer unit that is not shown, the information on time and date kept by the timer unit is stored in the memory unit 34 as the time and date information 43.

### 3. Control Operation on Subject-Side Terminal

### (1) Control Operation in Normal Times

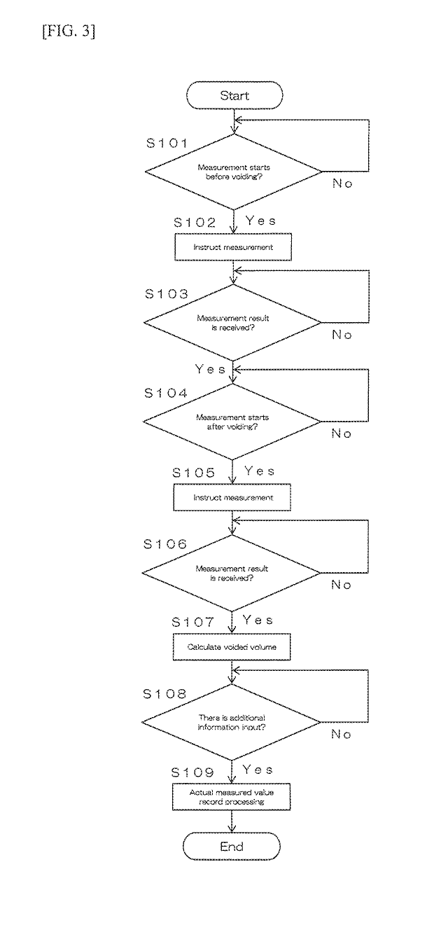

FIG. 3 is a flowchart showing one example of processing by the control unit 35 of the subject-side terminal 3 in normal times. Here, the normal times mean such times when the subject is not going out and when voiding of the subject does not involve any bowel movement.

With the voided volume management system 1, it is possible to manage information regarding voiding for a plurality of subjects. Further, a bar code is assigned to each of the subjects in order to identify the subject.

When voiding, the subject first turns power of the weight measurement unit 2 on. Further, the subject passes the assigned bar code over the camera 33.

Then, the bar code for the subject is read by the camera 33. Thereafter, information of the read bar code is input to the camera 33 as identification information. Further, the identification information input to the camera 33 is accepted by the identification information input accepting unit 357, and measurement for the subject corresponding to the accepted identification information starts.

On the operation display unit 32 of the subject-side terminal 3, various buttons are displayed in a selectable manner, such as a button for being outside, a button for measurement before voiding, and a button for measurement after voiding, for example.

Then, upon selection of the button for measurement before voiding by the subject (Step S101), by transmitting a signal to instruct measurement before voiding from the subject-side terminal 3 to the weight measurement unit 2, the weight measurement unit 2 becomes ready for measurement (Step S102).

Next, the subject steps on the weight measurement unit 2. Then, the weight of the subject is measured by the weight measurement unit 2, and information of a weight value that has been measured is transmitted to the subject-side terminal 3. Further, the subject-side terminal 3 receives the information of the weight value before voiding that has been transmitted from the weight measurement unit 2 by the communication unit 31 (Yes in Step S103).

Thereafter, the subject steps down the weight measurement unit 2 to have voiding in the lavatory. Further, the weight measurement unit 2 is turned to a standby state.

Then, upon selection of the button for measurement after voiding by the subject (Step S104), by transmitting a signal to instruct measurement after voiding from the subject-side terminal 3 to the weight measurement unit 2, the weight measurement unit 2 becomes ready for measurement (Step S105).

Next, the subject steps on the weight measurement unit 2. Then, the weight of the subject is measured by the weight measurement unit 2, and information of a weight value that has been measured is transmitted to the subject-side terminal 3. Further, the subject-side terminal 3 receives the information of the weight value after voiding that has been transmitted from the weight measurement unit 2 by the communication unit 31 (Yes in Step S106).

The voided volume calculation unit 352 calculates a voided volume based on the measurement result transmitted from the weight measurement unit 2, that is, the information of the weight value before voiding and the information of the weight value after voiding (Step S107). Specifically, the voided volume is calculated by subtracting the weight value after voiding from the weight value before voiding. In other words, a difference between the weight value before voiding and the weight value after voiding is calculated as the voided volume.

Then, an instruction to input additional information is displayed on the operation display unit 32 of the subject-side terminal 3. Specifically, the operation display unit 32 displays buttons in a selectable manner, respectively indicating whether or not the voiding includes a feces, evaluation of the voided volume, whether or not there is a desire to void before voiding, and whether or not there is urinary incontinence before voiding, as the additional information.

The button indicating whether or not the voiding includes a feces is a button to input whether or not there is a bowel movement during voiding, and one of a button indicating that the voiding includes a feces and a button indicating that the voiding includes no feces is displayed in a selectable manner, for example.

The buttons indicating evaluation of the voided volume are buttons to input a degree of the voided volume evaluated by the subject, and a plurality of buttons indicating large, medium, and small are displayed in a selectable manner, for example.

The buttons indicating whether or not there is a desire to void before voiding are buttons to input whether or not the subject has a desire to void before voiding, and a plurality of buttons indicating that there is a desire to void before voiding, and that there is no desire to void before voiding are displayed in a selectable manner, for example.

The buttons indicating whether or not there is urinary incontinence before voiding are buttons to input whether or not the subject has urinary incontinence before voiding, and a plurality of buttons indicating that there is urinary incontinence before voiding, and that there is no urinary incontinence before voiding are displayed in a selectable manner, for example.

For example, in a case in which in the voiding of the subject, there is a desire to void before voiding, there is no urinary incontinence before voiding, there is no bowel movement during voiding, and the subject feels the voided volume is normal, the subject selects the buttons indicating that there is no feces in voiding, indicating that the voided volume is normal, indicating that there is a desire to void before voiding, and indicating that there is no urinary incontinence before voiding.

When the additional information 44 is input in this manner (Yes in Step S108), the additional information input accepting unit 354 accepts the input additional information 44, and the additional information 44 is stored in the memory unit 34. It should be noted that in this case, the additional information 44 includes information indicating that the voiding is not performed during going out.

At this time, in the memory unit 34, the additional information 44 is recorded in association with other information. Specifically, in the memory unit 34, the voided volume information 42 calculated by the voided volume calculation unit 352, the time and date information 43 at which the voided volume is calculated, and the additional information 44 accepted by the additional information input accepting unit 354 are recorded in association with the identification information 41 accepted by the identification information input accepting unit 357. In the memory unit 34, these pieces of the associated information are recorded as the voiding information 40. Here, in this case, the voided volume information 42 is recorded in the memory unit 34 as an actual measured value (Step S109).

Thereafter, the subject steps down the weight measurement unit 2, and turns the power of the weight measurement unit 2 off.

This operation is repeated every time when the subject voids.

In this manner, the plurality of pieces of voiding information 40 are recorded in the memory unit 34. Further, the plurality of pieces of voiding information 40 recorded in the memory unit 34 are transmitted from the information output unit 351 to the cloud server 4 via the communication unit 31, and recorded in the cloud server 4 (cf. FIG. 1). Using the examiner-side terminal 5, the examiner is able to check the voiding information 40 that is recorded in the cloud server 4.

(2) Control Operation in a Case when Subject Goes Out

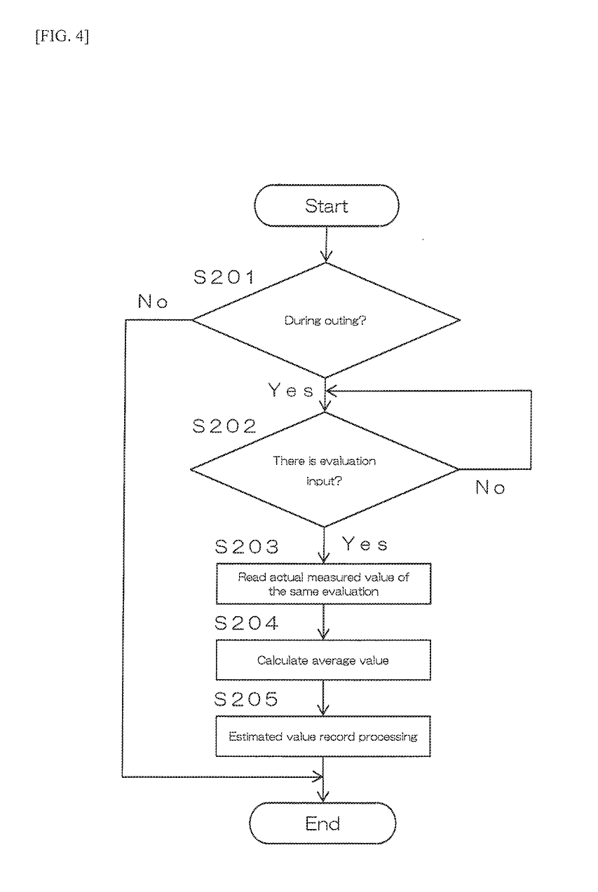

FIG. 4 is a flowchart showing one example of processing by the control unit 35 of the subject-side terminal 3 when the subject is going out.

The subject carries the subject-side terminal 3 when going out, and inputs required information to the subject-side terminal 3 when voiding. Alternatively, the subject may input information regarding voiding during going out to the subject-side terminal 3 after the subject comes home.

Specifically, the subject passes the assigned bar code over the camera 33 after voiding. Then, similarly to the case described above, the identification information of the subject is accepted by the identification information input accepting unit 357, and input of identification information starts.

Upon selection of a button indicating voiding during outing displayed in the operation display unit 32 by the subject (Yes in Step S201), buttons for evaluation of the voided volume are further displayed in the operation display unit 32 of the subject-side terminal 3.

The buttons for evaluation of the voided volume are buttons to input a degree of the voided volume evaluated by the subject, and are the same as the buttons for evaluation of the voided volume in normal times as the additional information described above. Specifically, the operation display unit 32 of the subject-side terminal 3 displays a plurality of buttons indicating large, medium, and small in a selectable manner, for example.

Upon selection of one of the buttons for evaluation of the voided volume by the subject (Yes in Step S202), the information reading unit 355 reads, out of the plurality of pieces of voiding information 40 recorded in the memory unit 34, a piece of the voiding information 40 having the same evaluation of the voided volume in the additional information 44 (Step S203). For example, when the medium button out of the buttons for evaluation of the voided volume is selected by the subject, the information reading unit 355 reads, out of the plurality of pieces of voiding information 40 for the same subject recorded in the memory unit 34, one or more pieces of the voiding information 40 having the additional information 44 indicating that the evaluation of the voided volume is medium.

If more than one piece of the voiding information 40 of the same evaluation is read, the estimated value record processing unit 356 calculates an average value based on these pieces of the voided volume information 42 in the voiding information 40 (Step S204). Then, the estimated value record processing unit 356 stores the calculated value in the memory unit 34 as an estimated value of the voided volume. Here, if only one piece of the voiding information 40 of the same evaluation is read, the voided volume information 42 in this piece of the voiding information 40 is stored in the memory unit 34 as the estimated value.

At this time, in the memory unit 34, information for the voided volume is recorded as the voided volume information 42 in association with other information. Specifically, in the memory unit 34, the voided volume information 42 calculated as the estimated value, the time and date information 43 at which the voided volume is calculated, and the additional information 44 indicating the voiding during outing are recorded in association with the identification information 41 accepted by the identification information input accepting unit 357. In the memory unit 34, these pieces of the associated information are recorded as the voiding information 40. Here, in this case, the voided volume information 42 is recorded in the memory unit 34 as the estimated value (Step S205).

(3) Control Operation in a Case when Subject has Bowel Movement

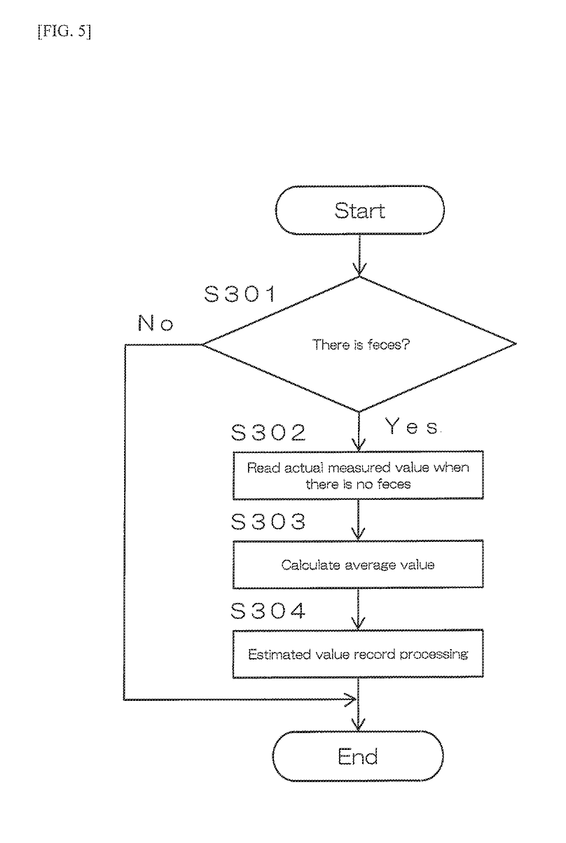

FIG. 5 is a flowchart showing one example of processing by the control unit 35 of the subject-side terminal 3 when the subject has a bowel movement.

If the subject has a bowel movement during voiding, the memory unit 34 records an estimated value as the voided volume information 42.

Specifically, in Step S108 of FIG. 3, if the button indicating that the voiding includes a feces in the operation display unit 32 is selected by the subject (Yes in Step S301), the information reading unit 355 reads, out of the plurality of pieces of voiding information 40 recorded in the memory unit 34, one or more pieces of the voiding information 40 having the additional information 44 indicating that there is no feces during voiding (the voiding information 40 whose voided volume information 42 indicates the actual measured value) (Step S302).

If more than one piece of the voiding information 40 is read, the estimated value record processing unit 356 calculates an average value based on these pieces of the voided volume information 42 in the voiding information 40 (Step S303). Then, the estimated value record processing unit 356 stores the calculated value in the memory unit 34 as an estimated value of the voided volume. Here, if only one piece of the voiding information 40 is read, the voided volume information 42 in this piece of the voiding information 40 is stored in the memory unit 34 as the estimated value.

At this time, in the memory unit 34, information for the voided volume is recorded as the voided volume information 42 in association with other information. Specifically, in the memory unit 34, the voided volume information 42 calculated as the estimated value, the time and date information 43 at which the voided volume is calculated, and the additional information 44 indicating that there is a feces during voiding are recorded in association with the identification information 41 accepted by the identification information input accepting unit 357. In the memory unit 34, these pieces of the associated information are recorded as the voiding information 40. Here, in this case, the voided volume information 42 is recorded in the memory unit 34 as the estimated value (Step S304).

### 4. Effects

(1) According to this embodiment, as illustrated in FIG. 2, the voided volume calculation unit 352 automatically calculates the voided volume of the subject, based on the information of the weight value before voiding and the information of the weight value after voiding. The memory unit 34 records the calculated voided volume of the subject as the voided volume information 42 in association with the additional information 44.

Therefore, it is possible to carry out detailed management of the information regarding voiding.

(2) Further, according to this embodiment, the additional information 44 includes the information on whether or not the voiding is carried out during outing, the information on whether or not the voiding includes a feces, the information on evaluation of the voided volume of the subject, the information on whether or not there is a desire to void before voiding, and the information on whether or not there is urinary incontinence before voiding.

Therefore, it is possible to carry out management of the additional information 44 useful for testing. Out of the additional information 44, the information on whether or not the voiding is carried out during outing and the information on whether or not the voiding includes a feces are useful as information indicating whether or not it is difficult for the voided volume calculation unit 352 to calculate the voided volume. Further, the information on evaluation of the voided volume of the subject is useful as information for associating the sense of the subject with an actual voided volume. In addition, the information on whether or not there is a desire to void before voiding, and the information on whether or not there is urinary incontinence before voiding are useful as information on conditions before voiding that can affect the test result.

(3) Moreover, according to this embodiment, as shown in FIG. 4 or FIG. 5, when an input of the information indicating that the voiding is carried out during outing, or the information indicating that there is a feces during voiding is accepted as the additional information 44 by the additional information input accepting unit 354, the information reading unit 355 reads the voiding information 40 for previous voiding recorded in the memory unit 34. The estimated value record processing unit 356 calculates the voided volume based on the voided volume information 42 in the past voiding information 40 that has been read by the information reading unit 355, and stores the calculated value in the memory unit 34 as an estimated value of the voided volume.

Specifically, when the calculation of the voided volume by the voided volume calculation unit 352 is difficult as in the case in which the voiding is carried out during outing, or in which the voiding includes a feces, the voided volume during outing, or the voided volume when there is a feces are recorded as the estimated value in the memory unit 34, based on the past voiding information 40 recorded in the memory unit 34.

Therefore, even when the calculation of the voided volume by the voided volume calculation unit 352, the information regarding the voided volume may be recorded in the memory unit 34, it is possible to perform detailed management of the information regarding voiding.

(4) Furthermore, according to this embodiment, as shown in FIG. 4, when in addition to the information indicating that the voiding is performed during outing, an input of evaluation for the voided volume by the subject are accepted by the additional information input accepting unit 354 as the additional information 44, the information reading unit 355 reads the voiding information 40 having the same evaluation of the voided volume.

Therefore, when the calculation of the voided volume by the voided volume calculation unit 352 is difficult, the information reading unit 355 reads, out of the past voiding information 40 recorded in the memory unit 34, the voiding information 40 including the voided volume information 42 whose voided volume is close to an actual voided volume based on the sense of the subject, and records the estimated value calculated by the estimated value record processing unit 356 in the memory unit 34.

Thus, even when the calculation of the voided volume by the voided volume calculation unit 352 is difficult, it is possible to perform accurate management of the information regarding voiding.

(5) Further, according to this embodiment, as shown in FIG. 4 and FIG. 5, the estimated value record processing unit 356 calculates an average value of the past voided volume information 42 that has been read by the information reading unit 355, and records the average value as the estimated value in the memory unit 34.

Therefore, the estimated value record processing unit 356 may calculate a value close to an actual voided volume using the past voided volume information 42 accumulated in the memory unit 34, and record the calculated value as the estimated value in the memory unit 34.

As a result, it is possible to perform accurate management of the information regarding voiding.

(6) Moreover, according to this embodiment, as illustrated in FIG. 2, the identification information input accepting unit 357 accepts an input of identification information from a plurality of subjects. The memory unit records the voided volume information 42 and the additional information 44, in association with the identification information 41 for each of the subjects for which input is accepted by the identification information input accepting unit 357.

Therefore, even when there is more than one subject, the voided volume information 42 and the additional information 44 may be managed for each subject.

As a result, it is possible to perform detailed management of the information regarding voiding for each subject.

(7) Furthermore, according to this embodiment, as illustrated in FIG. 1, the subject-side terminal 3 is configured separately from the weight measurement unit 2, and is able to perform communication with the weight measurement unit 2.

Therefore, the subject-side terminal 3 may be carried independently.

As a result, even when the subject is outside, it is possible to use the voided volume management system 1.

### 5. Modified Examples

According to the embodiment described above, the information reading unit 355 reads the voiding information 40 of the same evaluation as the input evaluation of the voided volume from the memory unit 34 when the subject goes out. However, the voiding information 40 that is read from the memory unit 34 by the information reading unit 355 when the subject goes out is not limited to this embodiment. Specifically, when the subject goes out, the information reading unit 355 may read all pieces of the voiding information 40 including the voided volume information 42 indicating a past actual measured value recorded in the memory unit 34. Then, the estimated value record processing unit 356 may calculate an average value based on the voided volume information 42 included in the plurality of pieces of voiding information 40 read by the information reading unit 355.

Further, according to the embodiment described above, the information reading unit 355 reads all pieces of the voiding information 40 including the voided volume information 42 indicating a past actual measured value recorded in the memory unit 34 when the subject has a bowel movement. However, the voiding information 40 that is read from the memory unit 34 by the information reading unit 355 when the subject has a bowel movement is not limited to this embodiment. Specifically, the information reading unit 355 may read the voiding information 40 of the same evaluation as the input evaluation of the voided volume from the memory unit 34 when the subject has a bowel movement. Then, the estimated value record processing unit 356 may calculate an average value based on the voided volume information 42 included in the plurality of pieces of voiding information 40 read by the information reading unit 355.

Moreover, according to the embodiment described above, the weight measurement unit 2 and the subject-side terminal 3 can communicate wirelessly. However, the weight measurement unit 2 and the subject-side terminal 3 may communicate by wire. It is also possible to configure the weight measurement unit 2 and the subject-side terminal 3 integrally in one piece.

Furthermore, according to the embodiment described above, the voiding information 40 recorded in the memory unit 34 is recorded in the cloud server 4 via the Internet N. However, the voiding information 40 recorded in the memory unit 34 may be recorded in a portable recording medium such as an SD card attached to the subject-side terminal 3 or a USB (Universal Serial Bus) memory.

Further, according to the embodiment described above, the information for the evaluation of the voided volume by the subject is selectable from three options of large, medium, and small. However, the information for the evaluation of the voided volume may be selectable from two or more options.

Moreover, according to the embodiment described above, the subject inputs information by manually operating the operation display unit 32 of the subject-side terminal 3. However, the subject may input information into the subject-side terminal 3 by sound.

What is claimed is:

1. A voided volume management system for management of voided volumes of a subject, comprising:
a weight measurement unit configured to measure a weight of a subject before and after voiding;
a voided volume calculation unit configured to calculate a voided volume based on a result of the measurement by the weight measurement unit;
an additional information input accepting unit configured to accept an input of additional information regarding voiding; and
a memory unit configured to record the additional information the input of which is accepted by the additional information input accepting unit, in association with the voided volume calculated by the voided volume calculation unit.

2. The voided volume management system according to claim 1, wherein
the additional information includes at least one piece of: information on whether or not the voiding is carried out during outing, information on whether or not the voiding includes a feces, information on evaluation of the voided volume by the subject, information on whether or not there is a desire to void before voiding, and information on whether or not there is urinary incontinence before voiding.

3. The voided volume management system according to claim 2, further comprising:
an information reading unit configured to read previous voided volumes recorded in the memory unit, when an input of information indicating that the voiding is carried out during outing or information indicating that there is a feces during voiding is accepted as the additional information by the additional information input accepting unit; and
an estimated value record processing unit configured to record, as an estimated value, one of a voided volume during outing and a voided volume when there is a feces, in the memory unit, based on the previous voided volumes read by the information reading unit.

4. The voided volume management system according to claim 3, wherein
when an input of the evaluation of the voided volume by the subject is accepted as the additional information by the additional information input accepting unit, along with the information indicating that the voiding is carried out during outing or the information indicating that there is a feces during voiding, the information reading unit reads previous voided volumes recorded in the memory unit in association with the same evaluation.

5. The voided volume management system according to claim 3, wherein
the estimated value record processing unit calculates an average value of the previous voided volumes read by the information reading unit, and records the average value as the estimated value in the memory unit.

6. The voided volume management system according to claim 1, further comprising:
an identification information input accepting unit configured to accept inputs of identification information from a plurality of subjects, wherein
the memory unit records a voided volume and additional information in association with the identification information of each of the subjects input of which is accepted by the identification information input accepting unit.

7. The voided volume management system according to claim 1, further comprising:
a terminal device configured separately from the weight measurement unit, and capable of communicating with the weight measurement unit, wherein
the additional information input accepting unit is provided in the terminal device.
